(19) United States
(12) Patent Application Publication
(10) Pub. No.: US 2005/0004563 A1
(43) Pub. Date: Jan. 6, 2005

(54) Apparatus and methods for sensing and cooling during application of thermal energy for treating degenerative spinal discs

(76) Inventors: N. Sandor Racz, Irving, TX (US); Tibor A. Racz, Dallas, TX (US)

Correspondence Address: TRASKBRITT, PC, P.O. Box 2550, Salt Lake City, UT, 84110, US

(21) Appl. No.: 10/611,667

(22) Filed: Jul. 1, 2003

(51) Int. Cl. A61B 18/18

(52) U.S. Cl. 606/34; 606/41

(57) ABSTRACT

Apparatus and methods for performing spinal disc lesioning procedures while monitoring the temperature near the spinal nerve roots. A needle containing a thermocouple and an injection bore may be placed between the disc and a nerve root. Temperature near the nerve root may be monitored during the lesioning procedure and if raised to a point where damage to the nerve could occur, the procedure may be stopped before damage is done. Coolant may be injected through the injection bore to lower the temperature near the nerve root. Additional dual purpose needles may be placed on the disc adjacent the opposite nerve root or in the spinal canal at the level of the disc for additional control. A hollow, flexible tip electrode needle may be used to repair and lesion the disc tissue. Prior to lesioning, the electrode may be stimulated to assess motor nerve response and avoid motor nerve damage.

### FIELD OF THE INVENTION

The present invention relates to the field of procedures for treating spinal pain. It is more particularly directed to procedures for treating degenerative disc conditions through the application of thermal energy to degenerative spinal discs while reducing the possibility of unintended injury to the spinal nerves and structures.

### BACKGROUND

Back pain may result from any of a number of underlying causes. Among these is damage to the spinal discs resulting from degenerative disease or injury. The spinal discs act as a cushioning structure between the individual vertebrae. Each spinal disc consists of a soft center, the nucleus pulposus (“nucleus”), concentrically surrounded by the fibrocartilage annulus fibrosus (“annulus”). Due to age or injury, the annulus can fray or tear which can result in discogenic back pain. Tears in the annulus may even allow seepage from the nucleus into the annulus or surrounding tissue, compounding the problem.

Procedures for lesioning the tissue of the annulus by application of heat were developed to treat this problem. These procedures include Intradiscal Electrothermal Annuloplasty (“IDET”), in which a heating element is introduced into the annulus or nucleus, and heated to 90 degrees C. to lesion the disc tissue. A number of difficulties were encountered using the IDET procedure. One attempt to resolve these difficulties has resulted in the development of radio-frequency (“RF”) thermal annuloplasty procedures, such as the DISCTRODE™ procedure. In the DISCTRODE™ procedure, the cannula is used to introduce a steerable, semi-rigid solid electrode through a cannula into the annulus. Electrical impedance is measured during introduction to avoid exiting the annulus or entering the nucleus. A temperature probe is then placed on the opposite external surface of the disc to measure temperature. Energy is then applied to the electrode to lesion the tissue with heat, while the external temperature is maintained at or below 45 degrees C.

During the development of the IDET and DISCTRODE™ procedures, it was believed that a change in collagen tissue occurred at 45 degrees C. to shortening the collagen fibers and lesioning the disc. It is now understood that this collagen fiber change does not take place in human tissue during these procedures. Instead, lesioning appears to disconnect nerve endings in the annulus, ending the pain of a torn annulus or degenerative process. There have been reported cases of paralysis occurring with these procedures that may result from the heating of the spinal cord or root nerves.

It would be desirable to provide a process or method for reducing the possibility of damage to the spinal nerves during application of thermal energy to the spinal discs. It would be further desirable to monitor and cool the temperature of tissue near the nerve roots, or in the spinal canal, or the epidural space, to protect the spinal nerves during lesioning.

### BRIEF SUMMARY OF THE INVENTION

The present invention is directed to devices and methods for performing spinal disc lesioning procedures while monitoring the temperature near the spinal nerve roots. A needle, such as a flexible tip needle, containing a thermocouple and an injection bore may be placed between the disc and a nerve root. The needle may be placed on the disc surface. Temperature near the nerve root may be monitored during the lesioning procedure, and if raised to a point where damage to the nerve could occur, the procedure may be stopped before damage is done. Coolant may be injected through the injection bore to lower the temperature near the nerve root. Additional dual purpose needles may be placed on the disc adjacent the opposite nerve root or in the spinal canal/epidural space at the level of the disc for additional control. A hollow, flexible tip electrode needle may be used to repair and lesion the disc tissue. Prior to lesioning, the electrode maybe stimulated to assess motor nerve response and avoid motor nerve damage. Alternatively, a curved or straight or blunt needle-electrode combination may be used.

### BRIEF DESCRIPTION OF THE DRAWINGS

The nature of the present invention as well as other embodiments of the present invention may be more clearly understood by reference to the following detailed description, to the appended claims, and to the several drawings herein, wherein:

FIG. 1 is a schematic top view of one embodiment of a procedure for achieving spinal disc lesioning, while monitoring temperature near the spinal nerve roots, in accordance with one embodiment of the present invention;

FIG. 2 is a side view of one embodiment of a monitoring needle useful in the procedure of FIG. 1, with an enlarged view section showing some details thereof;

FIG. 3 is a side view of the tips of some alternative embodiments of monitoring needles useful in the procedure of FIG. 1;

FIG. 4 is a side view of one embodiment of an electrode needle useful in the procedures of FIG. 1 and FIG. 6, with an enlarged view section showing some details thereof;

FIG. 5 is a side view of one embodiment of an electrode useful in the procedure of FIG. 1, with an enlarged view section showing some details thereof;

FIG. 6 is a schematic top view of one embodiment of a procedure for repairing a torn annulus, in accordance with the present invention; and

FIG. 7 is a schematic side view of one embodiment of a procedure for achieving spinal disc lesioning, while monitoring temperature near the spinal canal, in accordance with an embodiment of the present invention.

### DETAILED DESCRIPTION

The following describes some possible embodiments of this invention. It will be appreciated that the examples used herein are illustrative only and do not limit the invention.

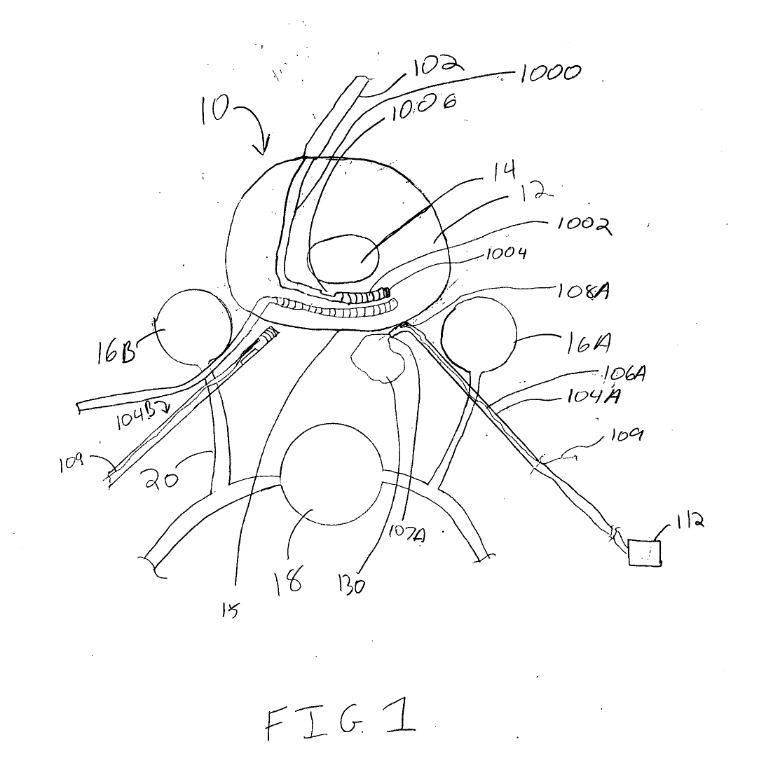

FIG. 1 represents a schematic top view of a spinal disc 10 and some of the surrounding nerve structures. Disc 10 consists of the nucleus 14 concentrically surrounded by the annulus 12. Two nerve roots 16, the sympathetic trunks, are adjacent the anterior surface of disc 10 on the right and left sides. The spinal cord 18 is directly posterior to disc 10, medial to the nerve roots 16. Spinal nerves 20 connect the spinal cord 18 and nerve roots 16 to one another and to the peripheral nervous system.

As shown in FIG. 1, the methods and processes of the present invention may be practiced to accomplish lesioning of disc 10 tissue while monitoring and adjusting the temperature around the nerve structures for protection. As an initial step, a curved blunt needle or spring tip needle (such as that disclosed in U.S. Pat. No. 6,371,943 to Racz et al. the disclosure of which is incorporated by reference herein) may be placed into or near the disc 10 and a contrast dye injected. This allows the remainder of the procedure to take place using fluoroscopy to monitor the placement of the surgical instruments and avoid the damaging the spinal and nervous tissue. A local anesthetic may be injected at the same time to allow the procedure to be conducted without pain to the patient.

A monitoring needle 104A is then placed on the surface 15 of disc 10 near nerve root 16A. Where possible, monitoring needle 104A may be placed directly between the surface 15 and nerve root 16A. Monitoring needle 104A includes a thermocouple 108A for measuring temperature. When the monitoring needle 104A is placed in position, the thermocouple 108A will measure the temperature near the nerve root 16A. Thermocouple 108A may be any commercially available thermocouple suitable for medical use and may be formed as an attached structure to monitoring needle 104A or may be passed down a bore thereof to the proper position. Communication lines 109 attach to thermocouple 108A and may be connected to a thermocouple monitoring system, or other read out device to allow a practitioner to monitor the temperature of the tissue.

Monitoring needle 104A may include an injection bore 106A with an injection opening 107A allowing fluids to be injected through the hollow monitoring needle 104A from an attached syringe or other fluid reservoir 112 system. It will be appreciated that while a single monitoring needle 104A including both an injection bore 106A and a thermocouple 108A may be used, a separate thermocouple and a single injection needle may be used where desired.

With the monitoring needle in place, the disc 10 may be lesioned as discussed further herein, or by using any known disc lesioning procedure, such as IDET or the DISCTRODE™ RF frequency lesioning. Using thermocouple 108A and the communications lines 109, the temperature near the nerve root 16A may be monitored, including the temperature of disc 10 surface 15. If the temperature approaches a predetermined level, the lesioning procedure may be halted. Once the temperature subsides, the procedure may be resumed. In this manner, disc lesioning may proceed while the nerve root 16A is protected from thermal harm.

Where the temperature of the disc surface 15 exceeds a desired range, a cooling fluid 130, such as a sterile saline may be injected through injection bore 106A and injection opening 107A to lower the temperature of the affected area. The cooling fluid 130 may be maintained in the reservoir 112 at a desired temperature to aid in this cooling action.

A second monitoring needle 104B may be inserted on the opposite side of the disc 10 near the opposite nerve root 16B allowing for temperature monitoring and control at multiple points across the disc 10. This allows for better monitoring and control over the entire procedure, especially where the lesioning procedure involves moving an electrode E to multiple positions inside the disc 10 or annulus 12.

Monitoring needles 104A and 104B may be any needle that can be inserted into the space between the nerve root 16 and disc 10, and are capable of monitoring the temperature of the disc surface in connection with a thermocouple 108. Suitable needles include insulated needles, uninsulated needles, injectable needles with side ports or open ends, blunt tip needles, bullet tip needles, curved needles, straight needles and flexible tip needles. Flexible tip needles, such as spring tip needles similar to those disclosed in U.S. Pat. No. 6,371,943 to Racz et al., may be particularly useful as they have an enhanced directional placement ability. Any additional structures or procedures needed to place the monitoring needles 104, such creating an incision to insert a non-cutting “pencil-point” or blunt tip needle, or introduction and insertion through a cannula may be used. Monitoring needle placement and the procedure may be carried out under fluoroscopy.

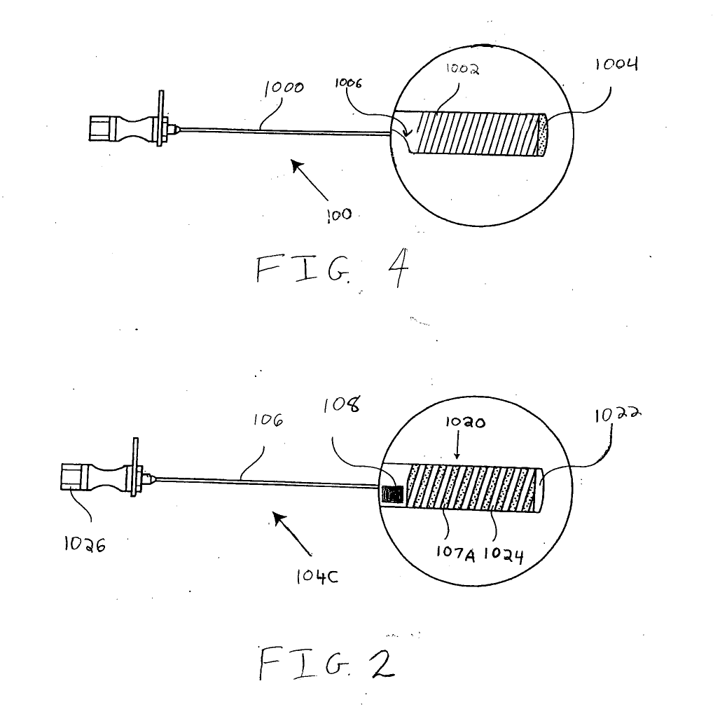

FIG. 2 depicts details of one embodiment of a flexible tip needle 104C that maybe used as a monitoring needle 104. In the depicted embodiment, a hollow shaft 106 has a distal flexible tip, such as spring tip 1020 and may have a blunt end 1022. Thermocouple 108 is mounted to, or contained within, the hollow shaft 106 for monitoring the tissue temperature. Where shaft 106 is constructed of an electrically conductive material, thermocouple 108 may electrically communicate with an attached monitoring system directly through the conductive portions of the shaft 106. Where desired, or where the shaft is formed from non-electrically conductive materials, communication lines 109 (FIG. 1) may be used. The details of tips of some other needles that may be used as monitoring needles 104, including a curved blunt end needle 104D, a straight blunt end needle 104E, a sharp end curved needle 104F, and a sharp end straight needle 104G are shown in FIG. 3. These needles may all include a thermocouple 108 and other structures as desired to practice the procedures of the present invention.

Injection opening 107 may be provided as a series of gaps 107A in the flexible sprig tip 1020. Gaps 107A may be provided as spaces between the windings of a spring 1024 forming the flexible spring tip 1020. In other embodiments, an opening or an open end may be provided. Any necessary structures for fastening the flexible needles 104C to a monitoring system for thermocouple 108 or a delivery system for cooling fluid 130 may be provided. One example is shown by the lockable needle hub 1026 of FIG. 2, through which the bore of the hollow shaft 106 may be accessed.

Also depicted in FIG. 1 is an electrode needle 100 useful for performing lesioning of the disc 10. For illustrative purposes two electrode needles 100A and 100B are depicted. Electrode needle 100A is introduced into the disc 10 and annulus 12 using a standard posterior approach. Electrode needle 100B is introduced into disc 10 and annulus 12 using an anterior approach. Recently, it has been shown that blunt needles do not perforate bowel tissues. Accordingly, it is possible to approach the anterior wall of disc 10 through the abdomen using an introducer needle device or cannula 102 with an interchangeable blunt tip and sharp tip protruding stylet. A blunt tip is used to pass through the abdomen without perforating bowel tissues and the sharp tip to pierce the disc 10 wall, allowing needle 100B to be introduced therein.

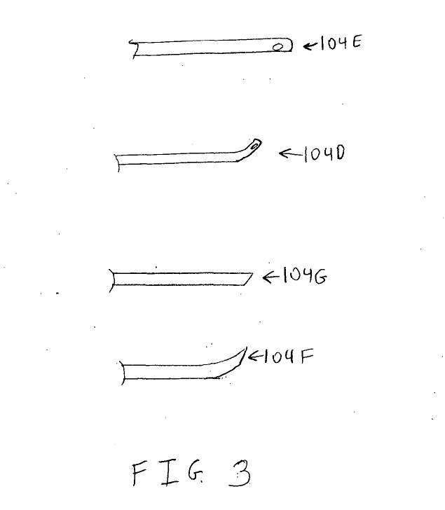

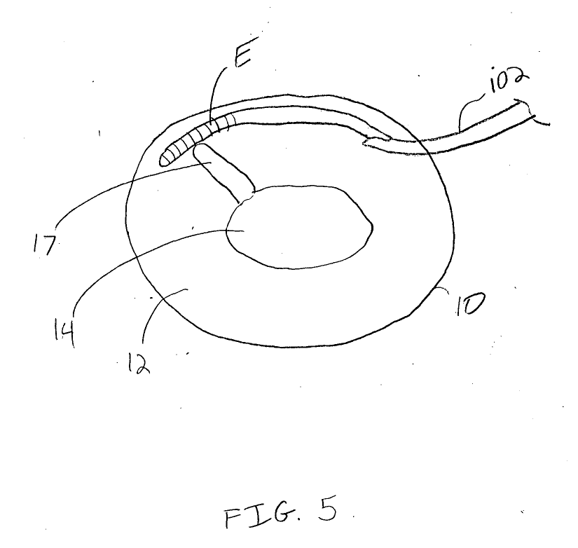

One embodiment of one suitable electrode needle is shown in further detail in FIG. 4. Electrode needle 100 may be a hollow flexible tip needle, including a hollow shaft 1000, a flexible tip 1002, such as a spring tip, and a conductive end 1004, which may be blunt. Conductive end 1004 may be constructed from any material having good conductive characteristics, such as gold, copper, steel and alloys thereof. Conductive end 1004 is connected to an external energy source, (e.g., an electromagnetic generator, such as an electrical, laser or radio-frequency generator) which transfers energy through a conductive wire, or alternatively, through internal conductive portions of the needle shaft 1000 and spring tip 1002 having insulated exteriors, and to the conductive tip 1004. Alternatively, where a larger area of contact is desired, only a portion of spring tip 1002 can be made or covered with a layer of insulative material so as to expand the area of conductivity.

After insertion, the electrode needle 100 may be stimulated to about 1 to about 2 volts, at about 2 to about 5 Hz, to reach the motor nerves at approximately 0.5 to 1.0 cm, prior to lesioning. Where motor nerve stimulation is observed at low voltage (below about 0.75 volts), the electrode needle 100 may be repositioned. In this way, the potential for motor nerve damage during lesioning can be further reduced. In embodiments, where lesioning is accomplished with electrodes other than electrode needle 100, this stimulation may be conducted with those electrodes. During a lesioning procedure, the electrode needle 100 may be used to check for motor stimulation between each increase in temperature, further protecting the nerves and structures throughout the procedure.

Electrode needle 100 may further include an opening, such as injection port 1006, allowing substances to be injected through the needle 100. Injection port 1006 maybe a side port, as depicted in FIG. 4, an opening in the distal end of the hollow shaft 100 or may comprise gaps in the flexible spring tip 1002. During disc lesioning, or when lesioning is halted, cooling fluid 130, such as saline can be injected directly into the disc. This procedure allows further control over the temperature of the lesioning procedure, as temperature is monitored at the disc surface 15 by the monitoring needle(s) 104.

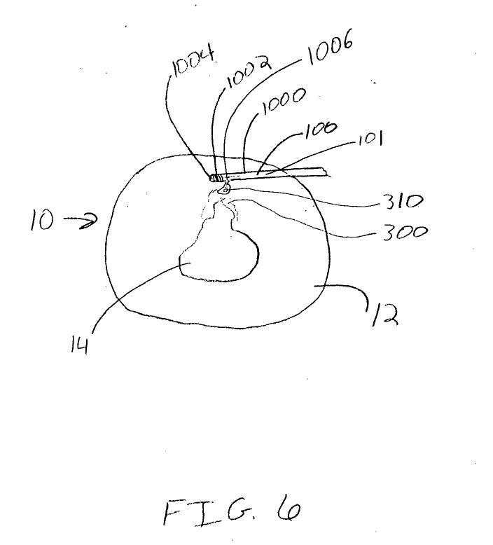

A cannula 102 contacts the disc 10 surface 15 and may even partially pass into the disc 10. Electrode needle 100 is passed through cannula 102 to enter annulus 12. With prior art disc lesioning procedures, heat may pass through a cannula that is electrically insulative, but thermally conductive. As lesioning proceeds, additional heat is passed into the surrounding tissues through the thermally conductive cannula. This heat can cause damage to the nerve roots 16 or other tissues. Accordingly, cannula 102 may be constructed of a material with thermally insulative properties, such as a polymer having both electrically and thermally insulative properties. Use of a thermally insulative cannula 102 reduces the heat transferred to the surrounding tissues during lesioning, allowing greater thermal control over the process. FIG. 5 depicts a more conventional disc lesioning electrode E, that is inserted through an insulative cannula D to repair a tear 17 in the annulus 12 of disc 10, that may be used in other embodiments of the present invention.

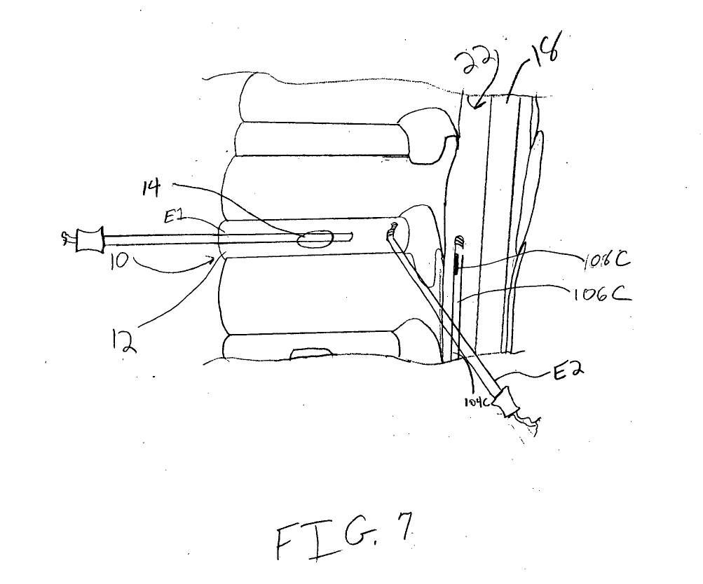

The use of a hollow electrode needle 100 provides additional functionality and flexibility to the disc lesioning and repair process. Turning to FIG. 6, a process for treating a torn annulus 12, where leakage from the nucleus 14 into surrounding tissue has occurred is depicted. Electrode needle 100 is directed to the area where a tear 300 in the annulus 12 fibrocartilage allows nucleus 14 leakage. A sealant material 310 is then injected into the tear 300 through the hollow electrode needle 100 and injection port 1006. The sealant material 310 fills in the tear 300 preventing further nucleus 14 leakage. The sealant material 310 may be any biologically compatible sealant material that is capable of injection through a needle. Fibrin-based products may be used as sealants. Other suitable sealant materials may include materials that are liquid for injection and then at least partially solidify to form a flexible sealant, or a gel-like sealant, upon the application of thermal energy. Once the tear 300 is filled with sealant material, such a sealant may be “set” in place by applying thermal energy through the conductive portion, such as conductive end 1004, of electrode needle 100. Any further disc lesioning that is needed may then be carried out. The temperature of this process may be monitored using monitoring needles 104, as discussed herein, to protect the spinal nerves.

Turning to FIG. 7, an additional monitoring needle 104C is shown in position to provide additional monitoring and control of temperature near spinal nerves during a disc lesioning procedure. Monitoring needle 104C may include the same features as monitoring needles 104A and 104B discussed previously herein, including a thermocouple 108C and a hollow bore 106C, as well as an injection opening 107C. Monitoring needle 104C may be inserted into the spinal canal 22, trans-sacrally at the ventral mid-spinal canal or transforaminally at the upper levels, or directed upwards thereto from a lower level. In some embodiments, a monitoring needle 104 may be inserted in the epidural space of, or adjacent to, the spinal canal 22. In such embodiments, the monitoring needle 104 may have a soft tip, such as a spring tip, and in addition to the structures discussed earlier herein, may also include a conductive structure 1023 (such as a conductive end), allowing electro-stimulation to be conducted therewith.

Disc lesioning may then occur using an electrode needle 100 (FIGS. 1 and 6), or in any other manner known to those of skill in the art. Electrodes E1 and E2 inserted in the annulus 12 represent a lesioning device in FIG. 7. Electrode E1 depicts an anterior approach and insertion, while electrode E2 depicts an posterior annulus electrode placement. It will be appreciated that only a single electrode may be used. As lesioning occurs, the temperature of the spinal canal 22 or epidural space may be monitored, if the temperature exceeds a desired level, lesioning may be halted, or a coolant solution maybe injected to cool the area. In this manner, the structures within the spinal canal, such as the cauda equina and the spinal cord maybe protected from thermal damage during the procedure. Where stimulation at a low voltage (such as about 0.75 Volts) is detected, to suggest posterior placement of the electrode (E2), fluid may be injected through the monitoring needle 104C to increase the size of the epidural space by volume displacement, further protecting the nerves and structures therein from thermal damage.

By injection of cold cooling fluid 130 through monitoring needles 104 A, B and C (where they are present), the temperature of affected portions of the annulus 12 may be elevated to accomplish lesioning, while disc 10 surface 15 temperature, or the temperature of the spinal nerves may be maintained within a desired range to reduce the occurrence of thermal damage. Using such methods, the intradiscal temperature may be raised to 65 degrees C., or even to 70 degrees C. (or potentially higher) while maintaining normal temperature near the spinal nerves. Inclusion of pre-lesioning motor nerve stimulation by the electrode reduces the possibility of nerve damage further.

It will be appreciated that the systems and components of the present invention may be offered as kits for surgical use. One example of a suitable kit could contain one or more monitoring and cooling needles 104. Another could include a hollow electrode needle 100 perhaps in connection with a suitable thermally insulative cannula 102. A third possible kit may include all these components together, all appropriate sized to function together and allow treatment of a particular spinal disc or discs. For example, one kit may be appropriately sized for lesioning of lumbar or thoracic spinal discs, while another is sized for treatment of cervical spinal discs. Each kit may further include appropriately sized introducing cannula, introducing needles or other components to provide all that is needed for a single disc lesioning procedure in one kit. Each kit may further include an openable package having a top, bottom, and sides defining a space for containing the surgical components. The package may keep the surgical components sterile until opened for use.

It will be apparent from the foregoing description that the apparatus and kits of the present invention may be readily manufactured and used by those of ordinary skill in the art, following the teaching of the present application. For example, needle shafts for monitoring needles and electrode needles may be made from polymeric materials, such as conventional medically useful plastics. These plastics may be extruded, injection molded or otherwise formed as known to those of ordinary skill in the art. Similarly, metal needles may be constructed using known methods for extrusion, rolling, forging and otherwise, all following the teachings of the present invention. Thermocouples, hubs, flexible tips and conductive ends may be attached to the shafts by compression, by bonding with suitable epoxies, by welding, or as otherwise known to those of ordinary skill in the art.

Although the present invention has been shown and described with respect to preferred embodiments, various additions, deletions and modifications that are obvious to a person skilled in the art to which the invention pertains, even if not shown or specifically described herein, are deemed to lie within the scope of the invention as encompassed by the following claims.

1. A method of protecting spinal nerves and structures during spinal disc heat lesioning procedures, said method comprising:
inserting a first hollow needle comprising a first thermocouple into a first space between the spinal disc and an ipsilateral root;
monitoring temperature near the ipsilateral root with the first thermocouple during spinal disc heat lesioning; and
reducing the temperature near the ipsilateral root in response to the monitoring to protect the ipsilateral root from thermal damage.

2. The method according to claim 1, wherein inserting a first hollow needle comprising a first thermocouple into a first space between the spinal disc and a ipsilateral root comprises contacting the first needle to a surface of the spinal disc.

3. The method according to claim 1, further comprising:
inserting a second hollow needle comprising a second thermocouple into a second space between the spinal disc and a contralateral root;
monitoring temperature near the contralateral root with the second thermocouple during spinal disc heat lesioning; and
reducing the temperature near the contralateral root in response to the monitoring to protect the contralateral root from thermal damage.

4. The method according to claim 1, further comprising:
inserting a dual purpose needle comprising a thermocouple and an injection bore into a spinal canal at a level equivalent to that of the disc;
monitoring temperature in the spinal canal with the thermocouple of the dual purpose needle during spinal disc heat lesioning, and
reducing the temperature in the spinal canal in response to the monitoring to protect the spinal nerves root from thermal damage.

5. The method according to claim 4, wherein reducing the temperature in the spinal canal in response to the monitoring to protect the spinal nerves from thermal damage comprises injecting a cooling fluid into the spinal canal through the injection bore of the dual purpose needle.

6. The method according to claim 1, further comprising:
inserting a dual purpose needle comprising a thermocouple and an injection bore into an epidural space at a level equivalent to that of the disc;
monitoring temperature in the epidural space with the thermocouple of the dual purpose needle during spinal disc heat lesioning, and
reducing the temperature in the epidural space in response to the monitoring to protect the spinal nerves root from thermal damage.

7. The method according to claim 6, wherein reducing the temperature in the epidural space in response to the monitoring to protect the spinal nerves from thermal damage comprises injecting a cooling fluid into the epidural space through the injection bore of the dual purpose needle.

8. The method according to claim 1, wherein reducing the temperature near the ipsilateral root in response to the monitoring to protect the ipsilateral root from thermal damage comprises halting spinal disc heat lesioning.

9. The method according to claim 1, wherein reducing the temperature near the ipsilateral root in response to the monitoring to protect the ipsilateral root from thermal damage comprises injecting a cooling fluid into the first space through the first hollow needle.

10. The method according to claim 1, wherein inserting a first hollow needle comprising a first thermocouple into a first space between the spinal disc and a ipsilateral root comprises inserting a spring tip needle into the first space between the spinal disc and the ipsilateral root.

11. A process for repairing a spinal disc annulus, said process comprising:
inserting a hollow needle comprising a conductive portion and an injection port into an annulus of the spinal disc;
injecting a sealant material into the annulus through the injection port to prevent a nucleus of the spinal disc from leaking into the annulus; and
lesioning a portion of the annulus with thermal energy applied through the conductive portion of the hollow needle.

12. The process according to claim 11, further comprising activating the sealant material to form a seal by applying thermal energy thereto through the conductive portion of the hollow needle.

13. The process according to claim 11, wherein injecting a sealant material into the annulus comprises injecting a fibrin-based sealant into the annulus.

14. The process according to claim 11, wherein inserting a hollow needle comprising a conductive portion and an injection port into an annulus of the spinal disc comprises inserting a hollow needle with a flexible spring tip having a conductive end into the annulus.

15. The process according to claim 11, wherein lesioning a portion of the annulus with thermal energy applied through the conductive portion of the hollow needle comprises applying RF thermal energy through the conductive portion of the hollow needle to lesion the annulus tissue.

16. The process according to claim 11, further comprising examining the spinal disc for motor nerve presence by applying electric current through the conductive portion of the hollow needle prior to lesioning.

17. A method for performing spinal disc lesioning, said method comprising:
inserting a hollow needle comprising a conductive portion into an annulus of a spinal disc;
lesioning a portion of the annulus with thermal energy applied through the conductive portion of the hollow needle.

18. The method according to claim 17, wherein inserting a hollow needle comprising a conductive portion into an annulus of a spinal disc comprises inserting a hollow needle with a flexible spring tip having a conductive end into the annulus.

19. The method according to claim 17, wherein lesioning a portion of the annulus with thermal energy applied through the conductive portion of the hollow needle comprises applying RF thermal energy through the conductive portion of the hollow needle to lesion annulus tissue.

20. The method according to claim 17, wherein inserting a hollow needle comprising a conductive portion into an annulus of a spinal disc comprises passing a thermally insulative cannula to the spinal disc and inserting the hollow needle into the annulus through the cannula.

21. The method according to claim 17, wherein inserting a hollow needle comprising a conductive portion into an annulus of a spinal disc comprises passing a blunt tipped introducing cannula to the spinal disc through the abdomen and inserting the hollow needle into the annulus through the cannula.

22. The method according to claim 17, further comprising monitoring temperature of a surface of the spinal disc during spinal disc lesioning and reducing the temperature in response to the monitoring to protect nerve structures near the spinal disc from thermal damage.

23. The method according to claim 22, wherein monitoring temperature of a surface of the spinal disc comprises inserting a dual purpose needle comprising a thermocouple and an injection bore to contact a surface of the spinal disc in a space between the spinal disc and a nerve root to monitor temperature of the disc surface near the nerve root.

24. The method according to claim 23, wherein reducing the temperature in response to the monitoring to protect nerve structures near the spinal disc from thermal damage comprises injecting a cooling fluid into the space between the spinal disc surface and the nerve root through the injection bore of the dual purpose needle.

25. The method according to claim 23, wherein reducing the temperature in response to the monitoring to protect nerve structures near the spinal disc from thermal damage comprises halting spinal disc lesioning to allow the spinal disc to cool.

26. The method according to claim 17, further comprising monitoring temperature near a spinal nerve structure during spinal disc lesioning and reducing the temperature in response to the monitoring to protect nerve structures near the spinal disc from thermal damage.

27. The method according to claim 26, wherein monitoring temperature near a spinal nerve structure comprises inserting a dual purpose needle comprising a thermocouple and an injection bore into the spinal canal at a level equivalent to that of the spinal disc to monitor temperature in the spinal canal near the spinal disc.

28. The method according to claim 27, wherein reducing the temperature in response to the monitoring to protect nerve structures near the spinal disc from thermal damage comprises injecting a cooling fluid into the spinal canal through the injection bore of the dual purpose needle.

29. The method according to claim 26, wherein monitoring temperature near a spinal nerve structure comprises inserting a dual purpose needle comprising a thermocouple and an injection bore into the epidural space near the spinal disc to monitor temperature in the epidural space.

30. The method according to claim 29, wherein reducing the temperature in response to the monitoring to protect nerve structures near the spinal disc from thermal damage comprises injecting a cooling fluid into the epidural space through the injection bore of the dual purpose needle.

31. The method according to claim 17, further comprising examining the spinal disc for motor nerve presence by applying electric current through the conductive portion of the hollow needle prior to lesioning.

32. A needle for monitoring a spinal disc lesioning procedure, comprising:
a hollow shaft;
an injection opening allowing access to a bore of the hollow shaft, configured for allowing injection therethrough; and
a thermocouple attached to the hollow shaft for monitoring temperature of the tissue surrounding the needle.

33. The needle of claim 32, further comprising a flexible tip disposed at a distal end of the hollow shaft.

34. The needle of claim 32, wherein the injection opening is formed by spaces between the windings of a coil spring forming the flexible tip.

35. A kit for performing a spinal disc procedure, comprising:
at least a first monitoring needle comprising a hollow shaft having a proximal and distal end, an injection opening allowing access to a bore of the hollow shaft, configured for allowing injection therethrough, a thermocouple attached to the hollow shaft for monitoring temperature of the tissue surrounding the at least a first monitoring needle;
an electrode needle, comprising an electrode shaft having a distal end and a proximal end, and a conductive portion for applying energy to spinal disc tissue.

36. The kit of claim 35, wherein the conductive portion of the electrode needle comprises a conductive blunt tip at a distal end of the electrode shaft.

37. The kit of claim 35, wherein the electrode shaft of the electrode needle comprises a hollow shaft.

38. The kit of claim 37, wherein the electrode needle further comprises an injection opening in fluid communication with a bore of the hollow shaft, allowing injection therethrough.

39. The kit of claim 35, further comprising a second monitoring needle comprising a hollow shaft having a proximal and distal end, an injection opening allowing access to a bore of the hollow shaft, and a thermocouple attached to the hollow shaft for monitoring temperature of the tissue surrounding the at least a first monitoring needle

40. The kit of claim 35, further comprising an openable package having a top, bottom, and sides defining a space for containing the at least a first monitoring needle and the electrode needle in a sterile manner.

41. An electrode needle for lesioning spinal disc tissue, said needle comprising:
a hollow shaft having a flexible distal tip and a proximal end; and
a conductive portion for applying lesioning energy to spinal disc tissue.

42. The electrode needle of claim 41, wherein the conductive portion comprises a conductive blunt tip at a distal end of the flexible distal tip.

43. The electrode needle of claim 41, further comprising an injection opening in fluid communication with a bore of the hollow shaft, allowing injection therethrough.

44. The electrode needle of claim 41, further comprising a thermally insulative insertion cannula.
